(19) United States
(12) Patent Application Publication
(10) Pub. No.: US 2016/0157736 A1
(43) Pub. Date: Jun. 9, 2016

(54) System and Methods for Assessment of Relative Fluid Flow Using Laser Speckle Imaging

(71) Applicants: Yu- Chih Huang, San Gabriel, CA (US); Owen Yang, Irvine, CA (US)

(72) Inventors: Yu- Chih Huang, San Gabriel, CA (US); Owen Yang, Irvine, CA (US)

(21) Appl. No.: 14/560,632

(22) Filed: Dec. 4, 2014

(51) Int. Cl. A61B 5/026; A61B 5/00

(52) U.S. Cl. CPC A61B 5/0261; A61B 5/7246; A61B 5/7278; A61B 5/742

(57) ABSTRACT

A system and method provide the capability to assess relative fluid flow. A light source is configured to generate a coherent light beam and direct the coherent light beam at an object. A detector is configured to receive light remitted from the object and output image data, and a controller is configured to receive the image data and calculate a relative fluid flow value based on a speckle contrast image of the image data.

### BACKGROUND

Anastomosis is a grafting procedure for providing additional or augmented blood flow to a body part or organ. Anastomosis is designed to improve blood flow, often by using a grafted blood vessel to circumvent an obstruction in the native blood flow system. Endovascular blood flow following anastomosis surgery is a significant concern regarding the success of the graft. During and/or following a surgical procedure, monitored blood flow in the grafted vessel can be used to assess the efficaciousness of the procedure.

### BRIEF DESCRIPTION OF THE DRAWINGS

One or more embodiments are illustrated by way of example, and not by limitation, in the figures of the accompanying drawings, wherein elements having the same reference numeral designations represent like elements throughout and wherein:

FIG. 1 is a schematic diagram of a system for assessing relative fluid flow according to one or more embodiments;

FIG. 2 is a diagram of an object according to one or more embodiments;

FIG. 3 is a depiction of a speckle image according to one or more embodiments;

FIG. 4 is a flow chart of at least a portion of a method of assessing relative fluid flow according to one or more embodiments;

FIG. 5 is a flow chart of at least a portion of a method of supporting a surgical procedure according to one or more embodiments;

FIG. 6 is a block diagram of a controller usable in accordance with one or more embodiments;

FIG. 7 is a diagram of an imaging object in accordance with one or more embodiments;

FIG. 8 is an example of speckle contrast images in accordance with one or more embodiments;

FIG. 9 is an example of speckle contrast values over time in accordance with one or more embodiments; and

FIG. 10 is an example of speckle contrast images and blood restoration values displayed on a display in accordance with one or more embodiments.

### DETAILED DESCRIPTION

The accompanying drawings, which are incorporated in and constitute a part of this specification, illustrate several embodiments of the invention and together with the description, serve to explain the principles of the embodiments described herein.

Laser Speckle Imaging (LSI) is a method of quantitatively measuring the optical scattering of particles over time. In an LSI application, coherent light incident to an object travels different path lengths before remitting from the surface of the object. Images collected from a detector over a finite sampling period yield a speckle image based on electromagnetic interference of the remitted light.

In some embodiments, the fluid is a liquid at the LSI measurement temperature. In some embodiments, the fluid is a gas at the LSI measurement temperature. In some embodiments, the LSI measurement temperature ranges from 2 to 315 K. In some embodiments, the temperature ranges from 308 to 314 K.

Although subject to many uses, determining fluid flow is useful in mechanical systems, e.g., hydrodynamic systems, and physiological systems, e.g., mammals, including humans, having blood flow.

If the collected light is captured using an exposure time longer than the fluctuation frequency of scattering particles, a blurring effect is produced in the speckle pattern. The degree of blurring can be quantified into a speckle contrast value that is proportional to the speed of a fluid flowing in the object. In a normalized function, a local speckle contrast value of zero value represents high flow and a value of one represents low flow, with intermediate values representing intermediate flow rates.

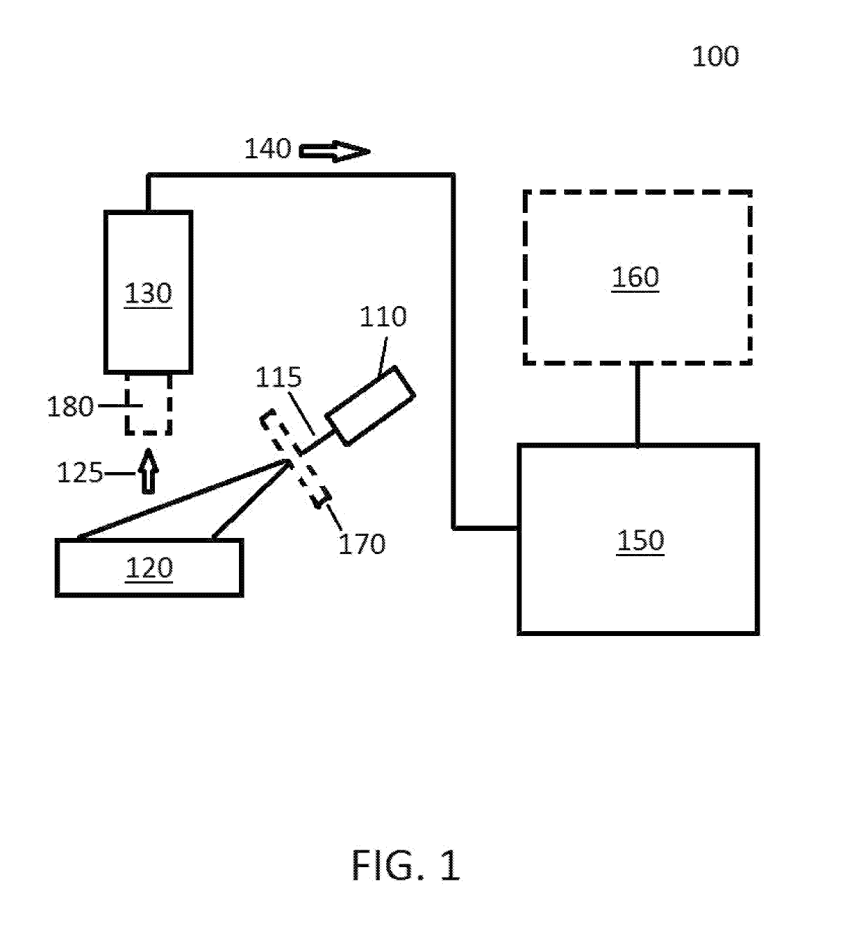

FIG. 1 is a diagram of a relative fluid flow assessment system 100 in accordance with one or more embodiments. System 100 comprises a coherent light source 110, a detector 130, and a controller 150. In some embodiments, system 100 comprises a display 160. In some embodiments, system 100 comprises an optical diffuser 170. In some embodiments, system 100 comprises a macro lens 180.

Coherent light source 110 is an apparatus capable of producing a coherent light beam 115 able to be directed at an object, e.g., object 120. In some embodiments, coherent light source 110 is a laser and coherent light beam 115 is a laser beam. In some embodiments, coherent light source 110 is a laser capable of producing coherent light beam 115 with a wavelength in the range of 300 nanometers (nm) to 1200 nm. In some embodiments, coherent light source 110 is a laser capable of producing coherent light beam 115 with a wavelength of 687 nm.

Object 120 is a substance, e.g., a manufacture or a composition of matter, in which one or more fluids flow. In some embodiments, object 120 is a living being in which blood flows. In some embodiments, object 120 is a human being in which blood flows. In some embodiments, object 120 is a human being in which blood flows through a native blood vessel and through a grafted blood vessel.

A portion of the light from coherent light beam 115 that reaches object 120 is remitted as remitted light 125. In some embodiments, portions of coherent light 115 travel different path lengths before remitting from the surface of object 120.

Although light source 110 is configured above object 120 in FIG. 1, in some embodiments, light source 110 is configured below object 120 such that object 120 is trans-illuminated by coherent light beam 115, and remitted light 125 is light that has passed through object 120. In various embodiments, light source 110 has a configuration relative to object 120 or angle of incidence on object 120 capable of producing remitted light 125.

Detector 130 is an apparatus capable of detecting remitted light 125. In some embodiments, detector 130 is configured to collect remitted light 125 over a predefined sampling period. In some embodiments, the predefined period is defined to yield a speckle pattern based on electromagnetic interference of remitted light 125. In some embodiments, object 120 has a substantially flat horizontal top surface and detector 130 is oriented vertically and positioned above object 120.

In some embodiments, detector 130 is a charge-coupled device (CCD) camera. In response to receiving remitted light 125, detector 130 is configured to output image data 140. In some embodiments, detector 130 is configured to output individual pixel data generated from detection of remitted light as image data 140. In some embodiments, detector 130 comprises a processor or other circuitry configured to process individual pixel image data into a format other than raw pixel data and output the processed data as image data 140.

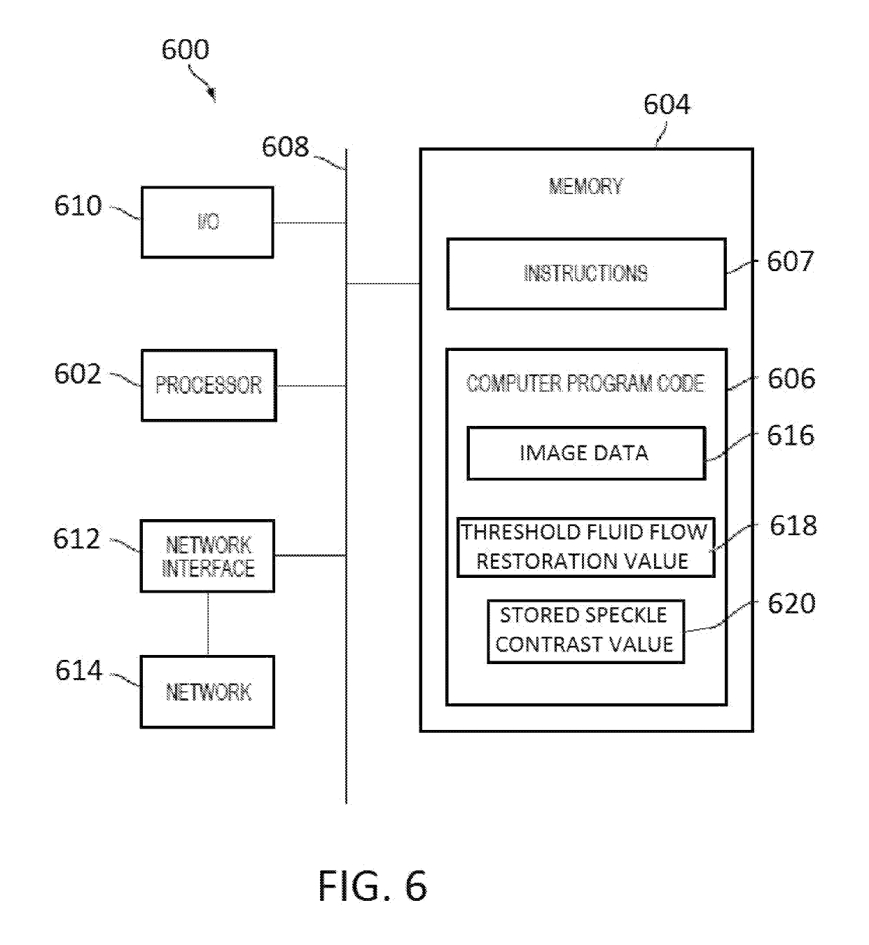

Controller 150 is an apparatus capable of receiving image data 140 and performing a calculation based on image data 140. In some embodiments, controller 150 comprises one or more processors configured to perform a calculation based on image data 140. In some embodiments, controller 150 comprises one or more application specific integrated circuits (ASIC) configured to perform a calculation based on image data 140. In some embodiments, controller 150 is a controller 600 (FIG. 6).

Display 160 is an apparatus capable of outputting an image to a user. In some embodiments, an image includes text. In some embodiments, display 160 comprises one or more of a cathode ray tube (CRT), a light emitting diode (LED) display, a liquid crystal display (LCD), a plasma display, or any other type of visual display technique. In some embodiments, display 160 is configured to output an audio signal.

Optical diffuser 170 is a device capable of diverging coherent light beam 115 to cover an expanded area. In some embodiments, optical diffuser is configured to expand coherent light source 115 to cover object 120.

Macro lens 180 is a device capable of modifying remitted light 125 before remitted light 125 is received by detector 130. In some embodiments, macro lens 180 is configured as one or more of a magnifier, photomultiplier, polarizer, or neutral density filter to have the capability of modifying remitted light 125. In some embodiments, macro lens 180 comprises adjustable aperture and magnification settings. In some embodiments, macro lens 180 is configured as a magnifier capable of magnifying remitted light 125 so that a speckle diameter of a speckle image is at least the width of two pixels of detector 130.

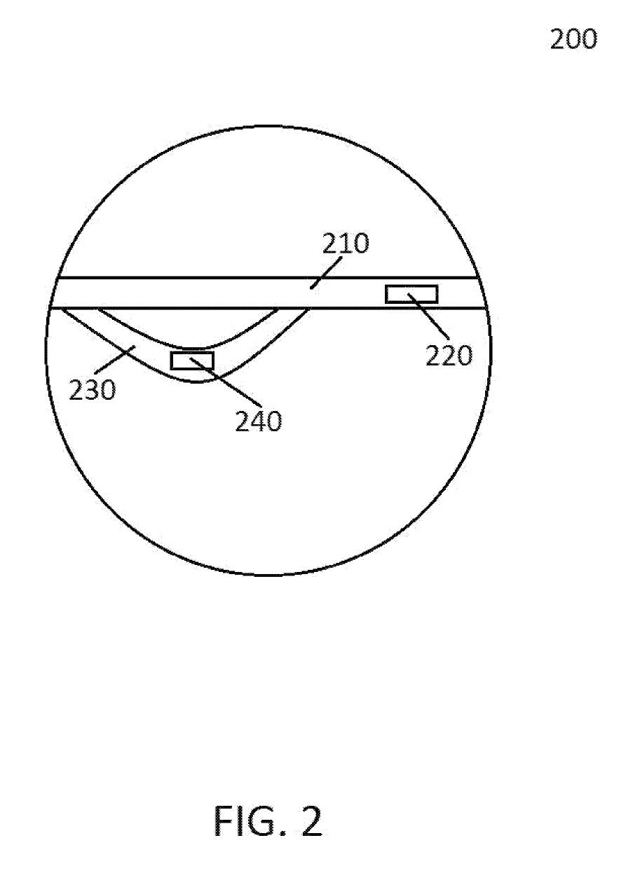

FIG. 2 is a diagram of an object 200, in some embodiments. Object 200 includes primary vessel 210 and secondary vessel 230. Primary vessel 210 and secondary vessel 230 contain fluid capable of flowing within one or both of primary vessel 210 and secondary vessel 230. In some embodiments, primary vessel 210 and secondary vessel 230 are blood vessels. In some embodiments, object 200 is an object 120 (FIG. 1).

In some embodiments, primary vessel 210 is a native blood vessel. In some embodiments, secondary vessel 230 is a grafted blood vessel. In some embodiments, primary vessel 210 is a native blood vessel and secondary vessel 230 is a blood vessel grafted to native blood vessel 210.

First area 220 is a portion of primary vessel 210 in which a fluid is capable of flowing. In some embodiments, first area 220 is a portion of a native blood vessel in which blood is capable of flowing. Second area 240 is a portion of secondary vessel 230 in which a fluid is capable of flowing. In some embodiments, second area 240 is a portion of a grafted blood vessel in which blood is capable of flowing. In some embodiments, second area 240 is a portion of a blood vessel grafted to a native blood vessel including area 220 in which blood is capable of flowing.

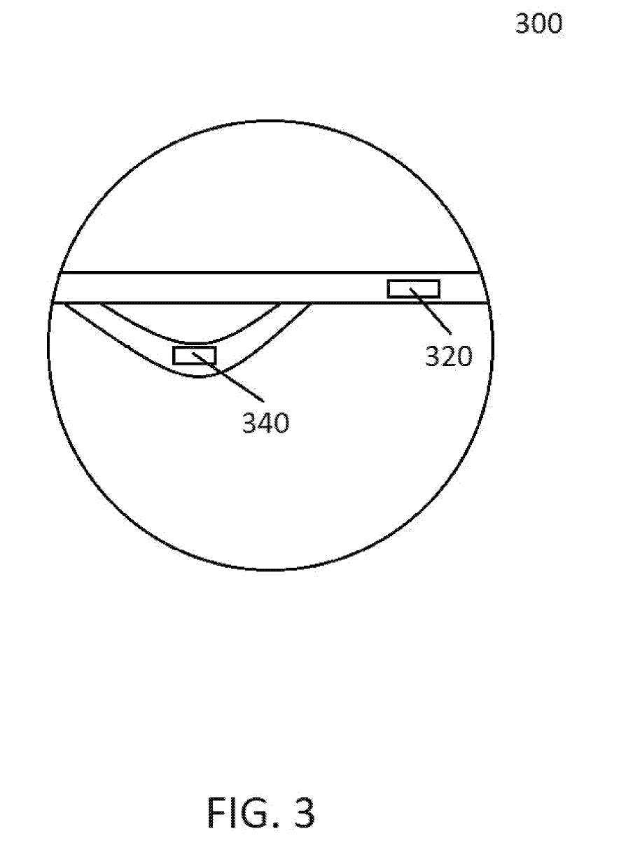

FIG. 3 is a depiction of a speckle contrast image 300. In some embodiments, speckle contrast image 300 is a speckle contrast image of object 200. In some embodiments, speckle contrast image 300 is displayed on display 160. Speckle contrast image 300 includes a first region 320 and a second region 340.

In some embodiments, first region 320 corresponds to first area 220. In some embodiments, first region 320 corresponds to first area 220 which is a portion of a native blood vessel in which blood is capable of flowing.

In some embodiments, second region 340 corresponds to second area 240. In some embodiments, second region 340 corresponds to second area 240 which is a portion of a grafted blood vessel in which blood is capable of flowing.

In some embodiments, quantifying speckle images consists of converting an entire raw image into a speckle contrast image by computing a local speckle contrast on a pixel-by-pixel basis as defined by:

K=σ/l (1)

where K is the local speckle contrast for a given pixel and σ and l are the standard deviation and average intensity, respectively, of remitted light for a local area in the image. The local area for the local speckle contrast calculation is the individual pixel and an area of pixels surrounding the local pixel.

In some embodiments, the local area is a 5×5 array of pixels with the given pixel at the center of the array. In some embodiments, the local area is a 7×7 array of pixels with the given pixel at the center of the array. In various embodiments, the local area is a circle or other symmetrical shape, or a non-symmetrical shape surrounding the given pixel.

The local area for speckle contrast calculations provides quantitative degrees of blurring where values that are closer to 0 represent values of higher flow and values closer to 1 represent values of lower flow. In some embodiments, controller 150 performs speckle contrast calculations.

In some embodiments, after calculating a speckle contrast image, two regions of interest taken from the speckle contrast image are needed to calculate a relative fluid flow value. In some embodiments, the first region of interest is first region 320, the second region of interest is second region 340, and a relative fluid flow value is calculated from first region 320 and second region 340. In some embodiments, first region 320 corresponds to a native blood vessel, second region 340 corresponds to a grafted blood vessel, and a relative fluid flow value is a blood flow restoration value.

In some embodiments, a relative fluid flow value is defined as:

V=K₁/K₂ (2)

where K₁ is the average speckle contrast of the first region of interest and K₂ is the average speckle contrast of the second region of interest. In some embodiments, controller 150 performs relative fluid flow calculations.

If the fluid flow in the first region of interest is higher than the fluid flow in the second region of interest, K₁ is lower than K₂ and the relative fluid flow value is less than one. If the fluid flow in the first region of interest is lower than the fluid flow in the second region of interest, K₂ is lower than K₁ and the relative fluid flow value is greater than one.

In some embodiments, one region of interest corresponds to a native blood vessel, another region of interest corresponds to a grafted blood vessel, and the relative fluid flow value is a blood flow restoration value. In these embodiments, the blood flow restoration value is defined as:

V=K_{N}/K_{G} (3)

where K_{N} is the average speckle contrast of the region of interest corresponding to the native blood vessel and K_{G} is the average speckle contrast of the region of interest corresponding to the grafted blood vessel. In some embodiments, controller 150 performs blood flow restoration calculations.

In blood flow restoration applications, the speckle contrast in the grafted blood vessel has the same approximate value as the speckle contrast in the native blood vessel, resulting in a blood flow restoration value of 1, in some embodiments. In some cases, blood flow in a grafted blood vessel has a lower flow (and thus a higher speckle contrast value) than a flow in a native blood vessel, which results in a blood flow restoration value less than 1, in some embodiments.

In clinical practice, varying degrees of blood flow restoration values are observable and various thresholds can be set for defining a successful outcome of a procedure such as anastomosis surgery.

In some embodiments, a relative fluid flow or blood flow restoration value is calculated from speckle contrast values from two regions of interest using an algorithm other than a ratio of contrast values.

In some embodiments, a single blood flow restoration value is used for supporting a surgical procedure. In some embodiments, a single blood flow restoration value is displayed on a display rather than an entire speckle contrast image.

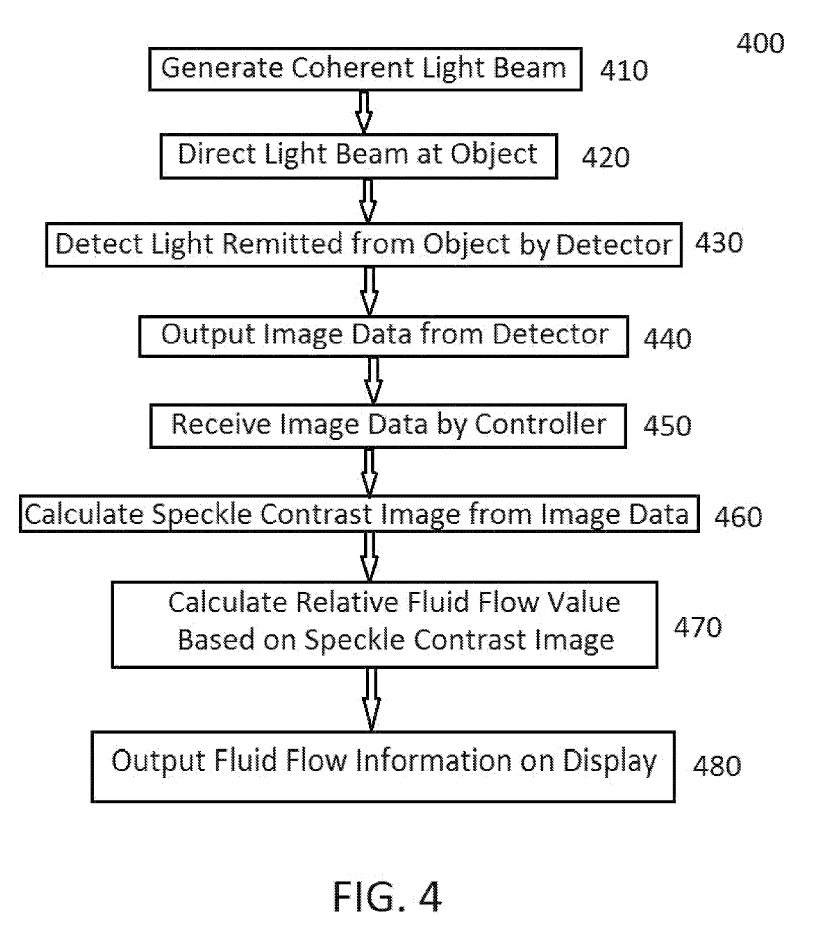

The present description also concerns a method of assessing relative fluid flow. FIG. 4 is a flow chart of at least a portion of a method 400 of assessing relative fluid flow according to one or more embodiments. Various embodiments include some or all of the steps depicted in FIG. 4. In some embodiments, the order of the steps varies from the order depicted in FIG. 4.

In some embodiments, method 400 includes step 410, in which a coherent light beam is generated. In some embodiments, coherent light beam 115 is generated by light source 110.

In some embodiments, method 400 includes step 420, in which the coherent light beam is directed at an object. In some embodiments, a coherent light beam is directed at an object by a light source. In some embodiments, a coherent light beam is directed at an object by a device other than a light source.

In some embodiments, coherent light beam 115 is directed at object 120. In some embodiments, coherent light beam 115 is directed at object 120 by light source 110. In some embodiment, coherent light beam 115 is directed at object 120 via optical diffuser 170.

In step 430, light remitted from an object is detected by a detector. In some embodiments, light remitted from an object travels directly to a detector. In some embodiments, light remitted from an object is magnified before being detected by a detector. In some embodiments, light remitted from an object is magnified by a macro lens before being detected by a detector. In some embodiments, remitted light 125 is detected by a detector 130.

In step 440, image data is output from the detector. In some embodiments, image data 140 is output from detector 130.

In some embodiments, individual pixel data generated from detection of remitted light is output by a detector. In some embodiments, individual pixel image data is reformatted by a processor or other circuitry prior to being output by a detector.

In step 450, image data is received by a controller. In some embodiments, image data is received by a controller 150.

In some embodiments, individual pixel data generated from detection of remitted light is received by a controller. In some embodiments, processed individual pixel image data is received by a controller.

In step 460, a speckle contrast image is calculated from image data. In some embodiments, a speckle contrast image is calculated from image data based on equation 1 as described above. In some embodiments, a speckle contrast image is calculated by controller 150.

In step 470, a relative fluid flow value is calculated based on a speckle contrast image. In some embodiments, a relative fluid flow value is calculated by controller 150. In some embodiments, a relative fluid flow value is calculated based on equation 2 as described above for first region 320 and second region 340. In some embodiments, a relative fluid flow value is a blood flow restoration value calculated based on equation 3 as described above for first region 320 corresponding to a native blood vessel and second region 340 corresponding to a grafted blood vessel.

In some embodiments, method 400 includes step 480, in which fluid flow information is output on a display. In some embodiments, fluid flow information is output on display 160. In some embodiments, fluid flow information comprises a relative fluid flow value. In some embodiments, fluid flow information comprises a speckle contrast image. In some embodiments, fluid flow information comprises a relative fluid flow value and a speckle contrast image.

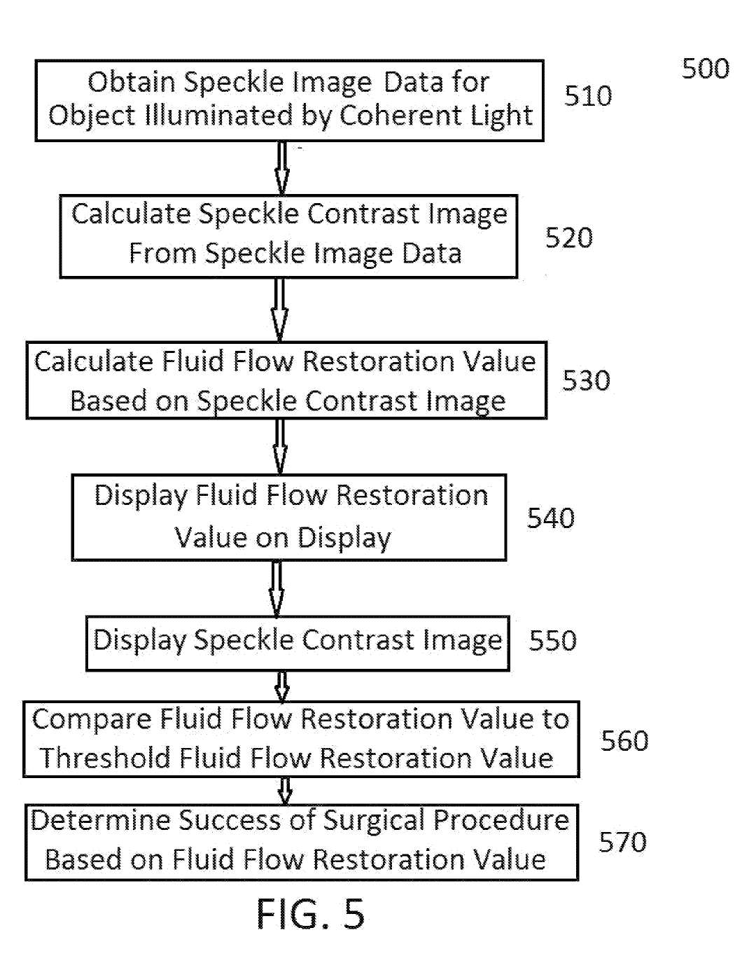

FIG. 5 is a flow chart of at least a portion of a method 500 of supporting a surgical procedure according to one or more embodiments. Various embodiments include some or all of the steps depicted in FIG. 5. In some embodiments, the order of the steps varies from the order depicted in

FIG. 5. In some embodiments, method 500 is used to provide feedback on fluid flow restoration during or after a surgical procedure such as anastomosis.

In step 510, speckle image data for an object illuminated by coherent light is obtained. In some embodiments, speckle image data for an object illuminated by coherent light is obtained by system 100 (FIG. 1). In some embodiments, speckle image data for an object illuminated by coherent light is obtained in accordance with method 400 (FIG. 4).

In step 520, a speckle contrast image is calculated from speckle image data. In some embodiments, a speckle contrast image is calculated from speckle image data based on equation 1 as described above. In some embodiments, a speckle contrast image is calculated by controller 150.

In step 530, a fluid flow restoration value is calculated from a speckle contrast image. In some embodiments, a fluid flow restoration value is calculated by controller 150. In some embodiments, a fluid flow restoration value is a blood flow restoration value calculated based on equation 3 as described above for first region 320 corresponding to a native blood vessel and second region 340 corresponding to a grafted blood vessel.

In some embodiments, a fluid flow restoration value is a blood flow restoration value calculated using equation 3 as described above based on a stored speckle contrast value for a native blood vessel obtained prior to a surgical procedure and a speckle contrast value for a blood vessel grafted as part of a surgical procedure.

In step 540, a fluid flow restoration value is displayed on a display. In some embodiments, a fluid flow restoration value is displayed on display 160. In some embodiments, a fluid flow restoration value displayed on a display is a blood flow restoration value. In some embodiments, a fluid flow restoration value is displayed on a display in real time.

In some embodiments, method 500 includes step 550, in which a speckle contrast image is displayed on a display. In some embodiments, a speckle contrast image is displayed on display 160.

In some embodiments, method 500 includes step 560, in which a fluid flow restoration value is compared to a threshold fluid flow restoration value. In some embodiments, a threshold fluid flow restoration value is a predetermined threshold fluid flow restoration value. In some embodiments, a fluid flow restoration value is a blood flow restoration value and is compared to a threshold fluid flow restoration value that is a threshold blood flow restoration value.

In some embodiments, displaying a fluid flow restoration value on a display includes displaying a threshold fluid flow restoration value on a display. In some embodiments, displaying a fluid flow restoration value on a display includes displaying a result of a comparison of a fluid flow restoration value to a threshold fluid flow restoration value on a display.

In some embodiments, method 500 includes step 570, in which a determination of success for a surgical procedure is based on a fluid flow restoration value. In some embodiments, a determination of success for a surgical procedure is based on a fluid flow restoration value displayed on a display. In some embodiments, a determination of success for a surgical procedure is based on a fluid flow restoration value and on a speckle contrast image displayed on a display.

In some embodiments, a determination of success for a surgical procedure is based on a comparison of a blood flow restoration value to a threshold blood flow restoration value. In some embodiments, a successful outcome for a surgical procedure is a blood flow restoration value above a predetermined threshold blood flow restoration value. In some embodiments, a successful outcome for a surgical procedure is a blood flow restoration value above a threshold blood flow restoration value that is determined after the start of the surgical procedure. In some embodiments, a surgical procedure is an anastomosis surgical procedure.

FIG. 6 is a block diagram of a controller 600 configured for assessing relative fluid flow in accordance with one or more embodiments. In some embodiments, controller 600 is similar to controller 150 (FIG. 1). Controller 600 includes a hardware processor 602 and a non-transitory, computer readable storage medium 604 encoded with, i.e., storing, the computer program code 606, i.e., a set of executable instructions. Computer readable storage medium 604 is also encoded with instructions 607. The processor 602 is electrically coupled to the computer readable storage medium 604 via a bus 608. The processor 602 is also electrically coupled to an I/O interface 610 by bus 608. A network interface 612 is also electrically connected to the processor 602 via bus 608. Network interface 612 is connected to a network 614, so that processor 602 and computer readable storage medium 604 are capable of connecting and communicating to external elements via network 614. In some embodiments, network interface 612 is replaced with a different communication path such as optical communication, microwave communication, inductive loop communication, or other suitable communication paths. The processor 602 is configured to execute the computer program code 606 encoded in the computer readable storage medium 604 in order to cause controller 600 to be usable for performing a portion or all of the operations as described with respect to relative fluid flow assessment system 100 (FIG. 1) or methods 400 (FIGS. 4) and 500 (FIG. 5).

In some embodiments, the processor 602 is a central processing unit (CPU), a multi-processor, a distributed processing system, an application specific integrated circuit (ASIC), and/or a suitable processing unit. In some embodiments, processor 602 is configured to receive image data via network interface 612. In some embodiments, processor 602 is configured to generate relative fluid flow information for transmitting to external circuitry via network interface 612.

In some embodiments, the computer readable storage medium 604 is an electronic, magnetic, optical, electromagnetic, infrared, and/or a semiconductor system (or apparatus or device). For example, the computer readable storage medium 604 includes a semiconductor or solid-state memory, a magnetic tape, a removable computer diskette, a random access memory (RAM), a read-only memory (ROM), a rigid magnetic disk, and/or an optical disk. In some embodiments using optical disks, the computer readable storage medium 604 includes a compact disk-read only memory (CD-ROM), a compact disk-read/write (CD-R/W), and/or a digital video disc (DVD). In some embodiments, the computer readable storage medium 604 is part of an embedded microcontroller or a system on chip (SoC).

In some embodiments, the storage medium 604 stores the computer program code 606 configured to cause controller 600 to perform the operations as described with respect to relative fluid flow assessment system 100 (FIG. 1) or methods 400 (FIGS. 4) and 500 (FIG. 5). In some embodiments, the storage medium 604 also stores information needed for performing the operations as described with respect to relative fluid flow assessment system 100, such as image data 616, a threshold fluid flow value 618, a stored speckle contrast value 620, and/or a set of executable instructions to perform the operation as described with respect to relative fluid flow assessment system 100.

In some embodiments, the storage medium 604 stores instructions 607 for interfacing with external components. The instructions 607 enable processor 602 to receive image data and generate operating instructions readable by external components to effectively implement the operations as described with respect to relative fluid flow assessment system 100.

Controller 600 includes I/O interface 610. I/O interface 610 is coupled to external circuitry. In some embodiments, I/O interface 610 is configured to receive instructions from a port in an embedded controller.

Controller 600 also includes network interface 612 coupled to the processor 602. Network interface 612 allows controller 600 to communicate with network 614, to which one or more other computer systems are connected. Network interface 612 includes wireless network interfaces such as BLUETOOTH, WIFI, WIMAX, GPRS, or WCDMA; or wired network interface such as ETHERNET, USB, IEEE-1394, or asynchronous or synchronous communications links, such as RS485, CAN or HDLC. In some embodiments, the operations as described with respect to controller 600 are implemented in two or more relative fluid flow assessment systems, and information such as image data are exchanged between different controllers 600 via network 614.

Controller 600 is configured to receive image data related to a speckle image from an external circuit. The information is transferred to processor 602 via bus 608 and stored in computer readable medium 604 as image data 616, threshold fluid flow restoration value 618, and/or stored speckle contrast value 620.

During operation, processor 602 executes a set of instructions to assess relative fluid flow as described with respect relative fluid flow assessment system 100 (FIG. 1) or methods 400 (FIGS. 4) and 500 (FIG. 5).

In a first example, an embodiment of a relative fluid flow assessment system was used to take images of clear silicone tubing with varying degrees of microlipid flow. The experiment demonstrated that calculated speckle contrast and relative fluid flow values are sensitive to a wide range of flow rates.

The relative fluid flow assessment system comprised a CCD camera, a macro lens, a laser source, an optical diffuser, and a processor. A 12-bit thermoelectrically cooled CCD camera (1,600×1,200 pixel resolution, Model 2000R, QIMAGING, Surrey, Canada) was used to obtain a raw speckle image remitted from the tubing. A 687-nm laser diode (40 mW power) was used to pass laser light through an optical diffuser to uniformly illuminate the tubing. By controlling the magnification and aperture of the macro lens, the speckle size was set to be at least the width of two camera pixels.

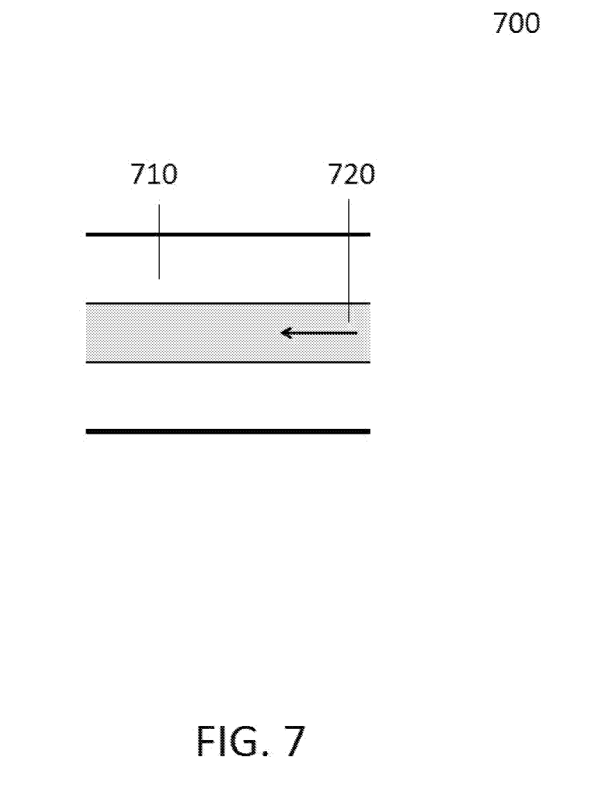

FIG. 7 is a diagram of imaging object 700 used in the experiment. Imaging object 700 consisted of a ⅛″ clear silicone tubing (MCMASTER-CARR, Elmhurst, Ill.) having tubing wall 710 and a plastic flow restrictor (not shown) capable of completely blocking the passage of a microlipid solution (NESTLE HEALTH SCIENCE, Florham Park, N.J.) 720 that mimics the optical properties of human blood in the visible and infrared spectrum.

The laser was shone upon the tubing and flow restrictor and the camera was placed approximately perpendicularly to the imaging surface.

The lateral velocity of microlipid solution 720 was varied manually between 0 and 2 mm/sec. For this example, the velocity of the solution was varied for six stages: (i) 2 mm/sec for approximately 20 seconds, (j) 0 mm/sec for 20 seconds, (k) 2 mm/sec for 20 seconds, (I) 0 mm/sec for 10, (m) 1 mm/sec for 10 seconds, and (n) 2 mm/sec for 20 seconds.

Images were captured during the experiment at a rate of approximately 9 frames per second. For a region of interest in the speckle images corresponding to a portion of the silicone tubing in which microlipid solution flow was controlled, local speckle contrast values were calculated, confirming that the speckle contrast decreased as the flow rate increased, and that samples could be taken with high temporal resolution.

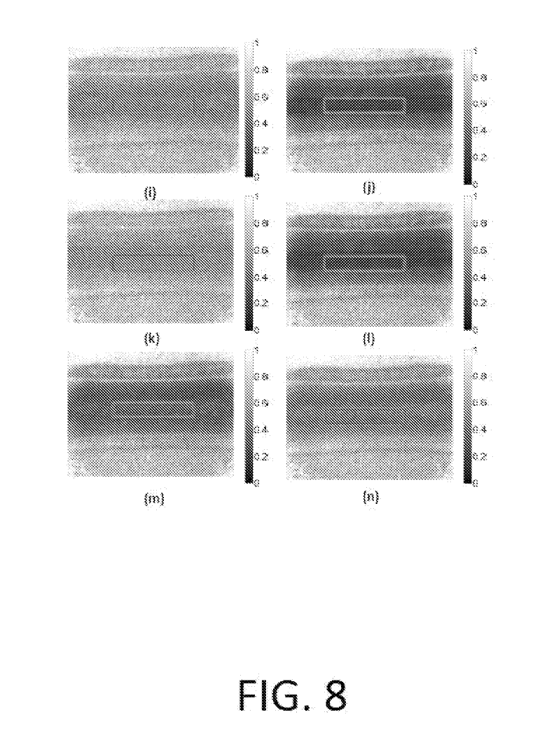

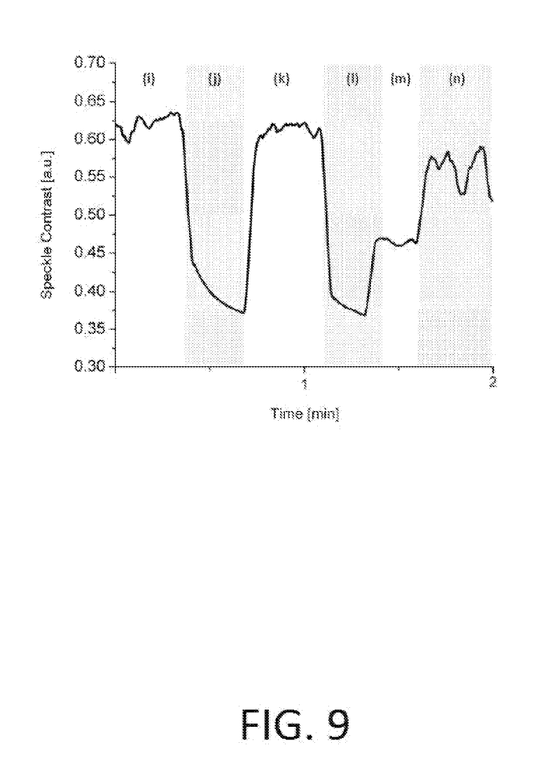

Speckle contrast images, converted from raw speckle image data, for the six stages are shown in FIG. 8, and speckle contrast values representing flow speeds for the six stages are shown in FIG. 9.

In a second example, an experiment was designed to mimic the use of the imaging system on a blood vessel that has been sutured onto a native blood vessel. The experiment was performed using the embodiment of the relative fluid flow assessment system described for the first example.

The flow restrictor was used to mimic the interface between two blood vessels that have been sutured together. A microlipid solution was used to flow through the tube and raw intensity images were taken with the CCD Camera under two conditions:
a) with the flow restrictor blocking passage of the solution from the right ‘original vessel’ to the left ‘new vessel;’ and
b) with the flow restrictor removed and the flow of solution passing from the original vessel' to the ‘new vessel.’

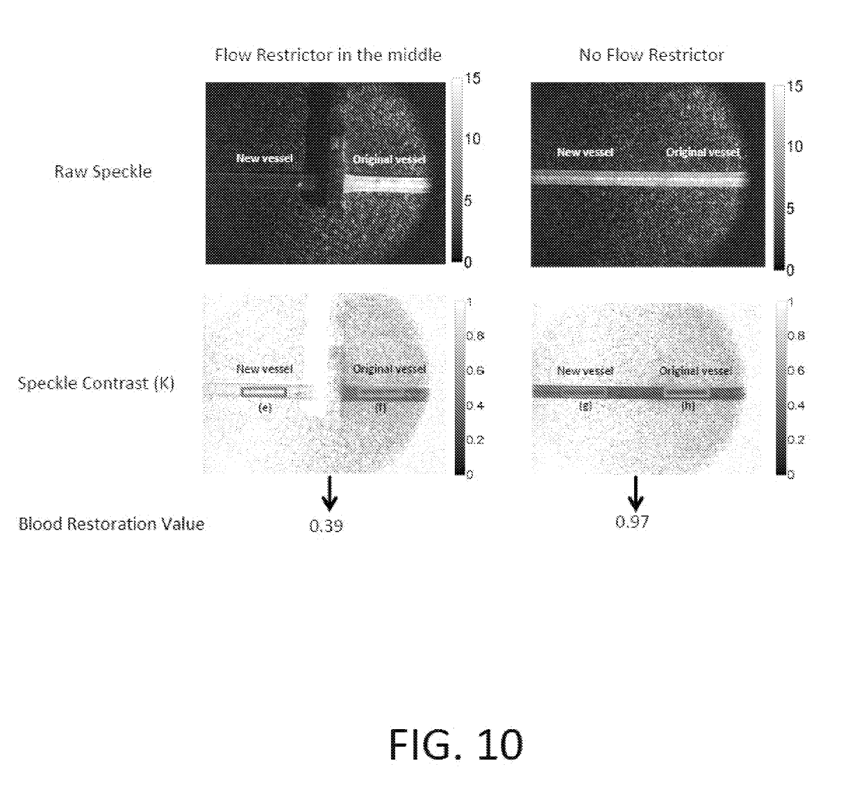

The raw speckle images with and without the flow restrictor are shown in FIG. 10. The corresponding speckle contrast images are also shown in FIG. 10. A single blood flow restoration value may be calculated by using the average of a group of pixels (in the blood vessel) of blood flow data prior to suture surgery and the blood flow pixels post suture surgery. The single blood flow restoration metric is calculated using equation 3 as described above. As shown in FIG. 10, the blood flow restoration metric before suture surgery is 0.39 and after suture surgery is 0.97. As expected, as the flow in the blood vessel after suture surgery becomes closer to the original vessel, the blood flow restoration metric tends towards 1.0.

The results were compared to the results of an approach known to each inventor in which a fluorescent dye was mixed with the microlipid solution and injected into the tubing. The approach used the dye, a light source to excite the dye, and a camera to pick up light from the dye once excited. In practice, this process is transient because the dye is usually filtered out of the human body within minutes. Image data collected by the camera produced a black and white image that provided qualitative feedback in the form of bright areas within the tubing.

The results from the relative fluid flow assessment system correlated with the qualitative results of the dye-based approach. In addition, the relative fluid flow assessment system provided ongoing feedback in the form of speckle contrast images and fluid flow restoration values without the use of a transient dye.

As demonstrated, the present disclosure provides sensitivity to varying degrees of flow rates and a blood flow restoration value for quantitative monitoring of relative blood flow rates, as opposed to a qualitative approach in which there is no differentiation between fluid flow rates. The present disclosure also provides the benefit of continuous blood flow monitoring by relying on the scattering of native red blood cells, which does not requiring invasive measures or the use of temporary markers.

In some embodiments, a system for assessing relative fluid flow comprises a light source configured to generate a coherent light beam and direct the coherent light beam at an object, a detector configured to receive light remitted from the object and output image data, and a controller configured to receive the image data and calculate a relative fluid flow value based on a speckle contrast image of the image data.

In some embodiments, a method of assessing relative fluid flow comprises detecting, by a detector, light from a coherent light beam remitted from an object, and outputting image data from the detector. The method further comprises receiving the image data by a controller and calculating, by the image data.

In some embodiments, a method of supporting a surgical procedure comprises obtaining a speckle contrast image of an object illuminated with coherent light, wherein the object comprises a native blood vessel and a grafted blood vessel. The method further comprises calculating, by at least one processor, a fluid flow restoration value based on the speckle contrast image, calculating the fluid flow restoration value comprising comparing a first metric value to a second metric value, wherein the first metric value is obtained from a first region in the speckle contrast image corresponding to the native blood vessel and the second metric value is obtained from a second region in the speckle contrast image corresponding to the grafted blood vessel, and displaying the fluid flow restoration value on a display.

Although the embodiments and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, and composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

Improved systems and methods for assessing restoration of blood flow are sought. Desirable features of one or more embodiments include non-invasive techniques, low sensitivity to patient motion, good spatial and temporal resolution, ease of use, low cost, and results that are easily accessed and interpreted.

It will be readily seen by one of ordinary skill in the art that the disclosed embodiments fulfill one or more of the advantages set forth above. After reading the foregoing specification, one of ordinary skill will be able to affect various changes, substitutions of equivalents and various other embodiments as broadly disclosed herein. It is therefore intended that the protection granted hereon be limited only by the definition contained in the appended claims and equivalents thereof.

What is claimed is:

1. A system for assessing relative fluid flow, the system comprising:
a light source configured to generate a coherent light beam and direct the coherent light beam at an object;
a detector configured to receive light remitted from the object and output image data; and
a controller configured to receive the image data and calculate a relative fluid flow value based on a speckle contrast image of the image data.

2. The system of claim 1, further comprising a display, wherein the controller is further configured to output one or more of the relative fluid flow value or the speckle contrast image on the display.

3. The system of claim 1, further comprising an optical diffuser configured to diffuse the coherent light beam.

4. The system of claim 1, further comprising a macro lens configured to modify the light remitted from the object so that a speckle diameter is at least the width of two pixels of the detector.

5. The system of claim 1, wherein the relative fluid flow value is based on a comparison of speckle contrast values from two regions in the speckle contrast image.

6. The system of claim 5, wherein the two regions correspond to a native blood vessel and a grafted blood vessel in the object.

7. A method of assessing relative fluid flow, the method comprising:
detecting, by a detector, light from a coherent light beam remitted from an object, and outputting image data from the detector;
receiving the image data by a controller; and
calculating, by the controller, a relative fluid flow value based on a speckle contrast image of the image data.

8. The method of claim 7, further comprising generating the coherent light beam by a light source and directing the coherent light beam at the object.

9. The method of claim 7, further comprising outputting one or more of the relative fluid flow value or the speckle contrast image on a display.

10. The method of claim 7, further comprising diffusing the coherent light beam on the object with an optical diffuser.

11. The method of claim 7, further comprising modifying the light remitted from the object so that a speckle diameter is at least the width of two pixels of the detector.

12. The method of claim 7, wherein calculating the relative fluid flow value comprises comparing local speckle contrast values from two regions in the speckle contrast image.

13. The method of claim 12, wherein the two regions correspond to a native blood vessel and a grafted blood vessel in the object.

14. A method of supporting a surgical procedure comprising:
obtaining a speckle contrast image of an object illuminated with coherent light, wherein the object comprises a native blood vessel and a grafted blood vessel;
calculating, by at least one processor, a first fluid flow restoration value based on the speckle contrast image, calculating the first fluid flow restoration value comprising comparing a first speckle contrast value to a second speckle contrast value, wherein the first speckle contrast value is obtained from a first region in the speckle contrast image corresponding to the native blood vessel and the second speckle contrast value is obtained from a second region in the speckle contrast image corresponding to the grafted blood vessel; and
displaying the first fluid flow restoration value on a display.

15. The method of claim 14, wherein the first fluid flow restoration value is a ratio of the first speckle contrast value to the second speckle contrast value.

16. The method of claim 14, further comprising displaying the speckle contrast image on the display.

17. The method of claim 14, wherein displaying the first fluid flow restoration value comprises displaying the first fluid flow restoration value in real time.

18. The method of claim 14, further comprising calculating a second fluid flow value by comparing a stored speckle contrast value to the second speckle contrast value, wherein the stored speckle contrast value is obtained prior to the grafted blood vessel being grafted to the native blood vessel.

19. The method of claim 14, further comprising comparing the first fluid flow restoration value to a threshold fluid flow restoration value.

20. The method of claim 19, further comprising determining a success of the surgical procedure based on a result of the comparison of the first fluid flow restoration value to the threshold fluid flow restoration value.
